(19) United States
(12) Patent Application Publication
(10) Pub. No.: US 2020/0129085 A1
(43) Pub. Date: Apr. 30, 2020

(54) WIRELESS TISSUE DIELECTRIC SPECTROSCOPY WITH RESONANT SENSORS

(71) Applicant: Iowa State University Research Foundation, Inc., Ames, IA (US)

(72) Inventors: Nigel Forest Reuel, Ames, IA (US); Sadaf Charkhabi, Ames, IA (US)

(21) Appl. No.: 16/667,671

(22) Filed: Oct. 29, 2019

Related U.S. Application Data
(60) Provisional application No. 62/752,602, filed on Oct. 30, 2018.

(51) Int. Cl. A61B 5/053; A61B 5/00

(52) U.S. Cl. CPC A61B 5/0531; A61B 5/6829; A61B 2503/08; A61B 5/0026; A61B 2505/07; A61B 5/6833

(57) ABSTRACT

Various embodiments disclosed relate to a resonator system that can be used to monitor status of tissue. A passive resonant sensor having an inductive element and a capacitive element can be implemented in a patch format and attached to tissue. The patch and resonant sensor can be structured such that, when contacting the tissue, dielectric of the contacted tissue contributes to capacitance of the resonant sensor to affect a resonant frequency of the resonant sensor. Additional apparatus, systems, and methods can be implemented with variations in a patch-resonant sensor structure.

### PRIORITY APPLICATION

This application claims the benefit of priority under 35 U.S.C. 119(e) from U.S. Provisional Application Ser. No. 62/752,602, filed 30 Oct. 2018, which application is incorporated herein by reference in its entirety.

### BACKGROUND

The U.S. health system is experiencing a significant burden, both economically and clinically, from chronic wound care, especially with an aging population and an increased prevalence of diseases such as obesity and diabetes. Chronic foot ulcers that result from diabetes lead to major tissue damage and possible amputation. These ulcers generally require constant upkeep, which can slow the healing progress if not treated or maintained properly. Current treatment typically involves the use of specialized footwear or support braces. In addition, constant application of moisture is vital to sustain the healing progress.

Acute wounds such as lacerations, punctures, abrasions, etc. also account for an additional burden. A key part of wound treatment is the systematic and routine assessment of wound status. Typically, best outcomes are observed when wounds are monitored weekly or even more frequently. Tools that could allow for accurate, at-home assessment of wounds, would off-load the burden from clinicians and greatly reduce the direct expense of clinic visits as well as resulting expenses, if problems are detected and managed earlier due to frequent monitoring at home.

Traditionally, wound assessment is performed by subjective, visual inspection by a trained clinician, sometimes enhanced by the use of a distance measuring tool, such as a ruler, to evaluate key dimensions of the wound. Imaging can be used to improve these traditional techniques by storing pictures for later comparison and for better quantification of wound area. There are many recent enhancements to imaging that enable improved diagnosis and tracking of wound healing, such as wound volume measurement via plenoptic lenses, tissue oxygenation via hyperspectral imaging, and local temperature assessment via infrared imaging. Another recent approach is to measure properties of the tissue local to the wound. Changes in tissue turgor and dielectric have both been exploited to determine wound healing phase.

### BRIEF DESCRIPTION OF THE FIGURES

The drawings illustrate generally, by way of example, but not by way of limitation, various embodiments discussed in the present document.

FIG. 1 is an illustration of an example flexible resonator printed on a polymer film, in accordance with various embodiments.

FIG. 2 is a block diagram of an example system architecture to wirelessly interrogate tissue using a patch-resonant sensor attached to the tissue, in accordance with various embodiments.

FIG. 3 shows an example output sensor readout from an algorithm to record signals from a vector network analyzer of the example system architecture of FIG. 2, in accordance with various embodiments.

FIG. 4 is an illustration of a side view of an example patch-resonant sensor included in a bandage for tissues, in accordance with various embodiments.

FIG. 5 shows a transmission scattering parameter magnitude response of a resonant sensor placed on tissue mimicking phantom models for increasing stages of ulcers, in accordance with various embodiments.

FIG. 6 shows plots of transmission magnitude response of a resonant sensor placed on tissue mimicking phantom models for laceration healing, in accordance with various embodiments.

FIG. 7 shows difference in reader response from healthy tissue of a dog and open wound on the dog, in accordance with various embodiments.

FIG. 8 is a flow diagram of features of an example method of monitoring tissue, in accordance with various embodiments.

FIG. 9 is a flow diagram of features of an example method of forming a passive patch-resonant sensor structure, in accordance with various embodiments.

FIG. 10 illustrates an example resonant sensor implemented in experiments to investigate the capability of the resonant sensor to detect bacteria on a surface, in accordance with various embodiments.

FIG. 11 shows experimental results in a plot of resonant frequency versus time for several optical density dilutions of B. subtilis cell culture, in accordance with various embodiments.

FIG. 12 shows additional experimental results in a plot of resonant frequency versus time for several optical density dilutions of B. subtilis cell culture, in accordance with various embodiments.

### DETAILED DESCRIPTION

The following detailed description refers to the accompanying drawings that show, by way of illustration, various embodiments of the invention. These embodiments are described in sufficient detail to enable those of ordinary skill in the art to practice these and other embodiments. Other embodiments may be utilized, and structural, logical, mechanical, and electrical changes may be made to these embodiments. The various embodiments are not necessarily mutually exclusive, as some embodiments can be combined with one or more other embodiments to form new embodiments. The following detailed description is, therefore, not to be taken in a limiting sense.

In current methods of assessing a wound, the assessment is typically conducted by exposing the wound for observation, examining the wound with a tool whose operation includes direct contact by the tool such as direct, wired contact by a tool that receives an input from wound by reading a signal generated from the wound. A tool which receives an input by reading a signal generated from an entity being probed, can be referred to as a reader. Currently there is no available contact-free method to assess the health of a bandaged wound.

In various embodiments, resonant biosensors can be implemental as a potential tool for real-time wound monitoring such as ulcer monitoring. Flexible resonators can be fabricated from thin sheets of conductive material, such as copper, deposited on a flexible substrate, such as a polyimide film. Flexible resonators can also be made by screen printing conductive ink on inexpensive substrates like polyethylene terephthalate (PET). Each resonator can have a set resonant frequency based on a coil geometry and dielectric medium in contact with the polyimide film. The resonator frequency and scattering profile change as the dielectric medium is altered. This response can be measured using a two loop antenna setup and vector network analyzer. A small, flexible, inexpensive, sterilizable resonator sensor in a patch format can be implemented to dynamically monitor wounds such as diabetic food ulcer stage wirelessly.

In various embodiments, a bandage or bandage material can be implemented with a flexible, passive patch that can be integrated into the bandage material, where the flexible, passive patch can include a device that can be used to wirelessly transduce changes in tissue dielectric over time. With the flexible, passive patch of the bandage placed on a wound, the changes in tissue dielectric result from material changes in the wound that accompany progression of the wound, since fluids of the wound change as the wound heals and as the wound worsens. A passive patch with its associated device is a patch and device structure that has no on-board power. The device of the passive patch can be realized by a resonant sensor having an inductive element and a capacitive element, where the resonant sensor is attached to or integrated into the patch with the patch along with the resonant sensor being attachable to tissue.

The patch and resonant sensor can be structured together such that, when contacting the tissue, dielectric of the contacted tissue contributes to capacitance of the resonant sensor to affect a resonant frequency of the resonant sensor. The resonant sensor may be implemented as the patch-resonant sensor structure. The contactless measurements using the patch-resonant sensor structure differs from the existing, contact-based dielectric measurement techniques in two significant ways. First, the patch-resonant sensor structure can be attached to the skin or embedded in a large bandage to allow for continuous monitoring and to reduce variability observed with contact devices caused by probe pressure. For example, pushing the reader of a contact-based tool harder on the skin can affect the measurement. Second, the patch-resonant sensor structure can be constructed such that the resonant sensor can be interrogated, contact-free through opaque, non-metallic materials thus allowing for assessment of wounds in bandages.

FIG. 1 is an illustration of an embodiment of an example flexible resonator 105 printed on a polymer film 103. A distance indicator is provided on the lower right hand side of FIG. 1. Flexible resonator 105 can be implemented as electrically conductive material in a coil structure. The electrically conductive material can be copper. Other electrically conductive material can be used. Flexible resonator 105 can be implemented as electrically conductive traces on polymer film 103. The electrically conductive trace can be encapsulated such that the encapsulation and the polymer film 103 protect the electrically conductive traces from shorting by wound fluids. The coil of flexible resonator 105 can be implemented as an Archimedean coil.

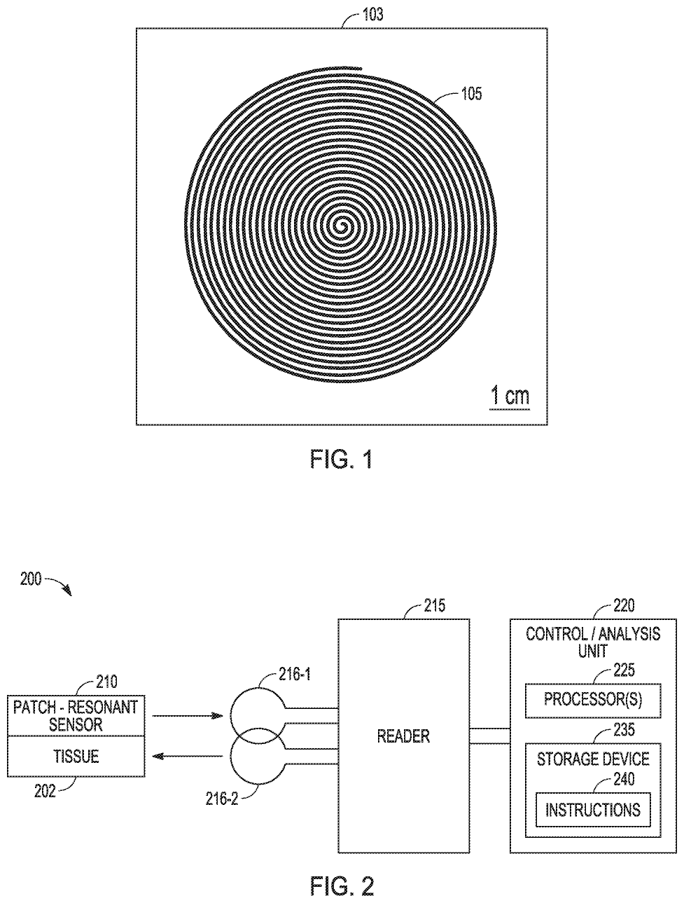

FIG. 2 is a block diagram of an embodiment of an example system architecture 200 to wirelessly interrogate tissue 202 using a patch-resonant sensor 210 attached to tissue 202. The tissue may be a wound with patch-resonant sensor 210 structured in a bandage. Patch-resonant sensor 210 on tissue 202 may be implemented as a resonant sensor attached to tissue 202. The resonant sensor of patch-resonant sensor 210 can be implemented as a resonator, such as an electrical circuit consisting of an inductor (L) and a capacitor (C) or an electrical circuit consisting of an inductor (L), a capacitor (C), and a resistor (R). An LC resonator is essentially an inductor and a capacitor placed in a parallel circuit that has a tuned frequency at which the circuit absorbs electromagnetic radiation. The resonator of patch-resonant sensor 210 can be a simple circuit that has an inductor in parallel with a capacitor. The inductor can be a looped or zig-zagged conductive trace with the capacitor being either a large, single element placed in parallel or can be composed of many small, capacitive regions that are present in the interstitial spaces of the inductor trace. The resonator of patch-resonant sensor 210 can be rendered into a sensor by engineering the circuit materials such that the tissue on which the sensor is attached contributes to the dielectric of the capacitor of the LC circuit. As tissue changes, such as with changes of a wound of the tissue, the dielectric of the tissue changes, which can cause a change in the capacitance of the LC circuit. Changes of the capacitance of the LC circuit in turn alters the resonant frequency of the LC circuit. Changes in the properties of LC circuits has been demonstrated for gas, pressure, temperature, and enzyme sensors. For various embodiments of tissue sensing such as monitoring a wound in tissue, a sensor can be placed on the wound site, where the resonant frequency of patch-resonant sensor 210 is affected by the local tissue dielectric. The local tissue dielectric can affect capacitance between printed circuit lines of patch-resonant sensor 210.

Patch-resonant sensor 210 can be wirelessly interrogated by a reader having a set of antennas including antenna 216-1 and antenna 216-2. Wireless interrogation is an electromagnetic probing of an entity without using electrical connections to the entity. A frequency spectrum can be transmitted from antenna 216-2 to patch-resonant sensor 210 and returned frequencies from patch-resonant sensor 210 can be received at antenna 216-1. Antenna 216-1 and antenna 216-2 are shown as two, co-planar loop antennas. As most wound locations typically do not allow for the wound to be placed between antennas, this co-planar, overlapping two-loop antenna design can be advantageous. Other configurations can be used. The generation of the frequency spectrum and processing of the returned frequencies can be performed by a reader 215.

Reader 215 can be a network analyzer. The network analyzer can be a standard vector network analyzer (VNA), which measures signals in terms of scattering parameters. The scattering parameters include parameters for reflected signal, S11, transmitted signal. S21, and reverse parameters, S22 and S12. The resonant frequency of patch-resonant sensor 210 can be monitored via a two-port vector network analyzer 215 that uses one port to transmit a frequency spectrum and the second port to monitor the returned frequencies. This arrangement measures the magnitude and phase of scattered and absorbed frequencies, namely the S11 and S21 scattering parameters. By recording these signals, clear resonant signal features, which are peaks and troughs, are observed and their modulations are observed for sensor readout. Sensor positional sensitivity of reader 215 can be addressed by embedding multiple patch-resonant sensor structures in a bandage, monitored at different start frequencies, proximal to a primary resonant sensor in the bandage such that the signals of the multiple patch-resonant sensor structures can be used for positional deconvolution. In addition, monitored signals from patch-resonant sensor 210 can be normalized based on their start frequency and extent of modulation rather than using fixed frequency thresholds. Alternatively, alignment marks can be placed on the bandage such that position of patch-resonant sensor 210 can be taken into account.

System architecture 200 can include a control and analysis unit 220, which can include an algorithm for tracking changes in resonant signatures from patch-resonant sensor 210. Control and analysis unit 220 can include one or more processors 225 and a storage device 235. Storage device 235 can store instructions for interrogating patch-resonant sensor 210, while patch-resonant sensor 210 is disposed on tissue 202 and can store data providing parameters for system architecture 200 and data from interrogating patch-resonant sensor 210. Storage device 235 can include a digital library of parameters for system architecture 200 and components of system architecture 200. Storage device 235 can be implemented as a group of memory devices to store data electronically. Such memory devices may be arranged as a distributed storage device, which may include remote memory devices accessed over the Internet or other network.

Storage device 235 can comprise instructions, which when executed by the one or more processors 225, that cause the system to perform operations to: interrogate the resonant sensor of patch-resonant sensor 210, with patch-resonant sensor 210 attached to tissue 202, at a number of different times using the set of antennas and the network analyzer; monitor the resonant frequency of the resonant sensor of patch-resonant sensor 210 from the interrogation at each time of the number of different times; and evaluate status of the tissue from the monitored resonant frequencies. Operations to evaluate the status of the tissue can include operations to identify changes in the monitored resonant frequency as a function of time and to correlate the identified changes to a healing status of the tissue. The operations to identify changes and to correlate the identified changes include identification of a shift of the monitored resonant frequency to lower frequencies with increase in time, from an initial interrogation of the resonant sensor with the patch attached to the tissue, as a healing of the tissue. In cases where a wound is in the form of an open cavity that fills in with fluid, going from a low air dielectric to a higher tissue dielectric, the resonant frequency can shift down. In wounds that begin with excess fluid, as this fluid is reduced, that is the tissue dries, which is accompanied by decreases in tissue dielectric, the resonant frequency can increase. The directional change in the resonant frequency can depend on the phase of the healing process and type of wound. The type of tissue being monitored can be used in evaluating the change in resonant frequency over time. A variety of tissue abnormalities (burns, open wounds, ulcers, inflammation, etc.) can change the tissue dielectric. If the tissue dielectric reduces, the resonant frequency can increase. If the tissue dielectric increases, the resonant frequency can decrease.

The operations can include operations to scan the resonant sensor to measure changes in morphology of the tissue, using the set of antennas including antennas 216-1 and 216-2 and reader 215 realized by a network analyzer to detect a phase and a magnitude of each of a S11 scattering parameter (reflection) and a S21 scattering parameter (transmission), which can provide four vectors for analysis. Both the S11 and S21 scattering parameters can be detected from patch-resonant sensor 210 with a two-port VNA, where S22 and S12 scattering parameters are neglected as they are symmetric to S11 and S21, respectively. Multiple signal features, such as peak frequency, width, and height can be used to perform principal component analysis (PCA) and deconvolute the data. Multivariate regression of the four vectors may also be used in analysis of the response.

Control and analysis unit 220 can be implemented to automate scanning of all scattering parameters (S11. S22, S12, and S21 magnitude and phase). Antennas can be coupled to a laptop for data acquisition, but reader 215, such as a VNA, can be operated without a monitor or graphical user interface (GUI) by controlling reader 215 with a single board computer such that a clinician can have a ‘point-and-click’ device that uses light emitting diode (LED) colors and blinking to communicate status. For example, a Linux-based single board computer can be used, though other operating systems can be used. Control and analysis unit 220 can analyze signal changes in polar coordinates to perform signal analysis utilizing both magnitude and phase of the scattering parameter. Other coordinate systems can be used. An algorithm in control and analysis unit 220 can track the modulation extent of the scattering parameter signals and normalize the response based on start signals, as each resonant patch implemented in patch-resonant sensor 210 will have a slightly different start frequency due to positioning on the wound and variations in fabrication. This normalized modulation extent can be used to correlate to wound health. The normalized modulation extent can be presented on a set scale such as (0 to 100).

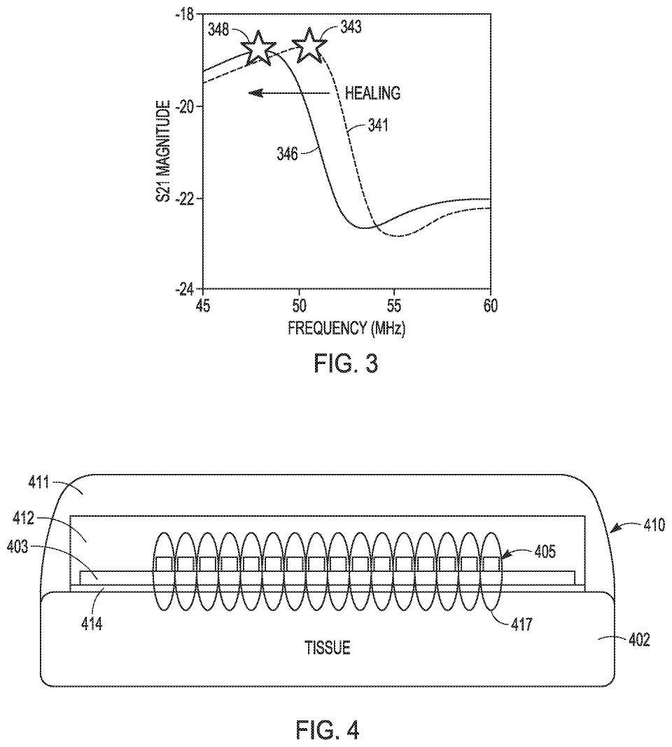

FIG. 3 shows an example output sensor readout from algorithm to record signals from VNA 215 of example system architecture 200 of FIG. 2. Curve 341 is an initial response for the resonant frequency presented by the magnitude of the S21 parameter as a function of frequency with a maximum at 343. Curve 346 is a response, after a selected time interval, for the resonant frequency presented by the magnitude of the S21 parameter as a function of frequency with a maximum at 348. Identification of a shift of a monitored resonant frequency to lower frequencies with increase in time, from an initial interrogation of the resonant sensor with the patch attached to the tissue, can be taken as a healing of the tissue. The shift can be identified with the shift from maximum magnitude 343 to maximum magnitude 348. In cases where a wound is in the form of an open cavity that fills in with fluid, going from a low air dielectric to a higher tissue dielectric, the resonant frequency can shift down. In wounds that begin with excess fluid, as this fluid is reduced, that is the tissue dries, which is accompanied by decreases in tissue dielectric, the resonant frequency can increase. The directional change in the resonant frequency can depend on the phase of the healing process and type of wound. The type of tissue being monitored can be used in evaluating the change in resonant frequency over time. A variety of tissue abnormalities (burns, open wounds, ulcers, inflammation, etc.) can change the tissue dielectric. If the tissue dielectric reduces, the resonant frequency can increase. If the tissue dielectric increases, the resonant frequency can decrease.

FIG. 4 is an illustration of a side view of an embodiment of an example patch-resonant sensor 410 included in a bandage 411 for tissues. Dotted ellipses 417 denote an electromagnetic field that is influenced by tissue 402 for an electromagnetic field that interrogates patch-resonant sensor 410 attached to tissue 402, in a pattern similar to application of system architecture 200 of FIG. 2. Patch-resonant sensor 410 may be implemented in a manner similar to or identical to implementation of patch-resonant sensor 210 in FIG. 2. Patch-resonant sensor 410 can include a resonator trace material 405 on a flexible substrate 403 where resonator trace material 405 includes conductive traces. Patch-resonant sensor 410 can include encapsulant material 412 to protect conductive traces of resonator trace material 405 from shorting by wound fluids. Patch-resonant sensor 410 can include a medical adhesive 414 to attach patch-resonant sensor 410 along with bandage 411 to tissue 402. Resonator sensors for patch-resonant sensors can be screen-printed on flexible substrates with conductive inks, printed via inkjet deposition, or can be etched from copper deposited on the substrate.

The sensor design for patch-resonant sensor 410 for application to bandaged wounds can include a number of variations. Resonator geometry, such as size and layout, and materials for encapsulant, substrate, adhesive, and conductive trace material can be selected from a database including these parameters. The database can store these parameters following an assessment of the manner in which these parameters have an effect of resonator performance, such as but not limited to tissue penetrating depth and maximum step-off distance through common bandage types, and robustness such as but not limited to impermeability, sterilizability, and flexure.

Testing of a tissue using a system architecture such as system architecture 200 of FIG. 2 was simulated using tissue mimicking phantoms (TMPs). Preliminary experiments were conducted with two sets of tissue-mimicking phantoms: diabetic foot ulcers as a pathogenesis model and laceration as healing model. For both sets of experiments, a resonator was probed with two, co-planar loop antennas attached to a two-port vector network analyzer at a step-off distance of 5 mm.

Recipes for phantom tissues and fluids (fat, muscle, blood, etc.) was obtained from literature that discussed using varying levels of oils, surfactants, and proteins to create hydrogels with dielectric properties that are similar to various dermal layers. Dielectric values of this materials were confirmed using four point impedance analyzer (data not shown). Red food coloring was added to the blood layer for enhanced visualization. These materials were cast in petri dishes with physiologically relevant layer depths to simulate healthy tissue and wounded tissue.

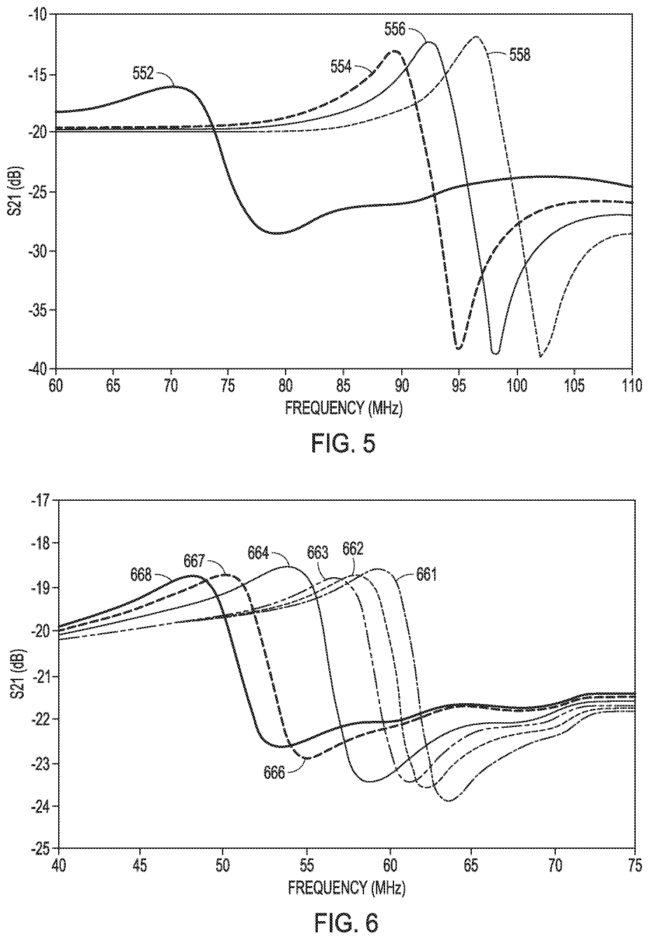

Stage 1 to 4 foot ulcers were simulated with TMPs with a resonant sensor placed on the surface of these models for the four stages. FIG. 5 shows a large increase in resonant frequency, as shown by a S21 magnitude response of a resonant sensor placed on TMP models for increasing stages of ulcers, as the ulcer progressed to deeper wounds in the simulation. Curve 552 is a plot of S21 scattering parameter as a function of frequency of a stage 1 foot ulcer associated with inflamed but unbroken. Curve 554 is a plot of S21 scattering parameter as a function of frequency of a stage 2 foot ulcer associated with visible dermis. Curve 556 is a plot of S21 scattering parameter as a function of frequency of a stage 3 foot ulcer associated with subcutaneous fat. Curve 558 is a plot of S21 scattering parameter as a function of frequency of a stage 4 foot ulcer associated with visible muscle. Curves 552, 554, 556, and 558, starting with curve 552, show pathogenesis with respect to foot ulcers.

Likewise, a laceration model was designed from TMPs that presented seven stages of wound healing. In this case, the epidermis was simulated using a thin chamois leather with the laceration cut, and the laceration size was reduced as the wound ‘healed.’ A stepwise decrease in resonant frequency was observed as the wound model ‘healed.’ FIG. 6 shows plots of S21 magnitude response of resonant sensor placed on TMP models for laceration healing. Curve 661 is a plot of S21 scattering parameter as a function of frequency associated with hemostasis. Curve 662 is a plot of S21 scattering parameter as a function of frequency associated with inflammation. Curve 663 is a plot of S21 scattering parameter as a function of frequency associated with a proliferation 1. Curve 664 is a plot of S21 scattering parameter as a function of frequency associated with a proliferation 2. Curve 666 is a plot of S21 scattering parameter as a function of frequency associated with a proliferation 3, which is for the most part the same as curve 667. Curve 667 is a plot of S21 scattering parameter as a function of frequency associated with remodeling. Curve 668 is a plot of S21 scattering parameter as a function of frequency associated with healed tissue. Curves 661, 662, 663, 664, 666, 667, and 668, starting with curve 661, show a healing progression.

In various embodiments, resonators, having different geometries, can be fabricated with a rapid-prototype, etching method. In such a method, a resonator design of inductor and capacitor is drawn on copper coated polymer with an indelible marker. For example, the polymer may be. Pyralux. This drawing masks the copper coil when placed in an etchant bath and the mask is washed off using acetone. Conductive metals other than copper can be used to construct the resonator as well as using different modalities of printing to the substrate, for example, sputtering followed by etching, screen printing, and inkjet printing.

Resonator designs can be open-circuit Archimedean coils, as shown in FIG. 1. Open-circuit Archimedean coils of a copper inductor with capacitance provided by dielectric material have been found to demonstrate strong S21 and S11 scattering response from such a resonant design. An encapsulant can be spin-coated onto the bare side of the resonator, which the side that is different from the side of the resonator coupled to a flexible substrate. Substrate refers to the material on which the conductive traces of the resonator are printed. Adhesives are used to bind the resonator sensor to a bandage and/or onto skin, such that it does not move between measurements and is held on the skin or wound dressing at a constant pressure. Such an adhesive can be attached to the substrate.

A digital library of parameters can be stored in a database that provides data, from testing, on different sizes and layouts of the coil for the resonant sensor. The data can also can be collected for the library that illustrates the effect of length and pitch size on performance and robustness of components as well as differences in layout such as but not limited to elliptical shape and variable pitch size. The effect of material selection on resonator performance and robustness can also be tested and stored in the digital library. Algorithms can be implemented using the stored data to determine the best combination of encapsulants, substrates, adhesives, and conductive metals as components for flexible resonant sensors to be integrated under or into a bandage. This determination of combinations can used be to select the appropriate patch-resonant sensor structure for a wound, as different wounds have different three-dimensional sizes. Such components can include parylene, epoxies, waxes, acrylics, and other materials as encapsulates in the digital library. Components can include PET, polyethylene-naphthalate (PEN), polyimide (PI), paper, and other material as substrate materials in the digital library. Components can include epoxies, surgical tape, cyanoacrylate glue, surgical glues, and other materials as adhesive materials in the digital library. Components can include etched copper, screen printed silver, inkjet printed silver, screen printed copper, and other conductive materials as metal traces for inductor elements in the digital library.

Other data can be included in a digital library. This digital library can include data that assess the effect of geometry and material changes of various combinations components for a patch-resonant sensor structure. Tissue penetration depth can be determined by creating a panel of TMPs that have a thin layer of higher dielectric (simulated bloody tissue or edema) embedded below varying thicknesses of lower dielectric (simulated fat tissue). Resonant sensor candidates of different combinations can be placed on the TMPs, and a depth at which the dielectric of ‘wounded’ tissue can no longer be detected can be determined. A limit of detection (LOD) for each combination of components can be set at the point where a shifted signal, such as a shifted signal discussed with respect to FIG. 3, falls below three times the variation of the signal caused by noise, which is typically an established LOD metric for most sensors. Another performance parameter that can be stored in the data library is a maximum step-off distance through common bandage types. From preliminary studies with a wound on a dog, it was observed that a combination of Telfa® pads, cast padding, conforming stretch gauze, and flexible cohesive bandage can be used to cover the wound. These components can be varied in layer thickness depending on the surgeon and wound location. Bandage composition and thickness can be varied in different testing, where determination of the LODs, set at signal strength equal to three times the signal noise, can be stored in the digital library.

A digital library can include data regarding sensor robustness as a measure of performance. The data can include, but is not limited to, data regarding sterilizability, impermeability to fluids, and flexure tolerance of different combinations of components for a patch-resonant sensor structure. The effect of sterilization can be determined in testing by subjecting candidate resonators to an established autoclave regimen used for surgical instruments and then observing the resonant frequency signal while submerged under 10× phosphate-buffered saline (PBS) buffer solution. The PBS buffer solution is a water-based salt solution containing disodium hydrogen phosphate, sodium chloride, and, in some formulations, potassium chloride and potassium dihydrogen phosphate. The PBS buffer solution helps to maintain a constant pH. The osmolarity and ion concentrations of the PBS buffer solution can match those of the human body. In this manner, the effect of the autoclave on the integrity of different encapsulants, different substrates, and different combination of encapsulants and substrates can be assessed and stored in the digital database.

A water-tight seal is important for wireless interrogation by a patch-resonant sensor structure application. Preliminary animal studies have shown that a bandaged wound is a fluid-rich environment. If such fluid penetrates across the conductive coil of the attached patch-resonant sensor structure, the resonant sensor of the patch-resonant sensor structure can short and no signal to monitor will be produced by the patch-resonant sensor structure. Testing of sealing structures can be monitored for the patch-resonant sensor structure being tested for any breakthrough of fluid, which is evidenced by a change in resonant frequency. These experiments can be made in duration of multiple days and can be include multiple patch-resonant sensor structures tested in parallel with a Bayonet Neill-Concelman (BNC) connector cable multiplexer arrangement with a single VNA. Additional data on flexure tolerance can be stored in the digital library by physically flexing patch-resonant sensor structures to a tight (0.5 cm) radius of curvature repeatedly, for example ten times, and recording occurrence of any break down in the resonator signal strength.

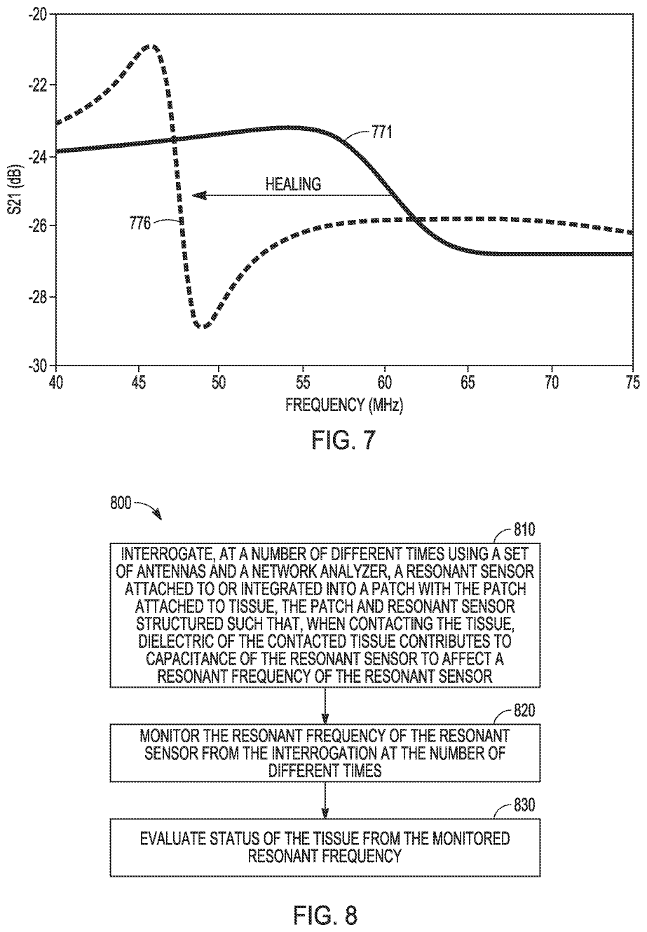

A preliminary animal study was conducted with a dog presented with a large wound on the top surface of the paw. This wound was cleaned and covered with a 0.5 mm thick polyurethane foam pad. The resonant sensor was then placed on the pad. The resonant sensor response was recorded after the initial surgery and then after ten days of recovery, showing a large shift in resonant frequency when measured at the same site. FIG. 7 shows difference in reader response from healthy tissue of the dog, curve 776, and open wound on dog, curve 771.

FIG. 8 is a flow diagram of features of an embodiment of an example method 800 of monitoring tissue. Method 800 may be a processor implemented method. At 810, at a number of different times using a set of antennas and a network analyzer, a resonant sensor attached to or integrated into a patch with the patch attached to tissue is interrogated. The patch and resonant sensor can be structured such that, when contacting the tissue, dielectric of the contacted tissue contributes to capacitance of the resonant sensor to affect a resonant frequency of the resonant sensor. At 820, the resonant frequency of the resonant sensor is monitored from the interrogation at the number of different times.

At 830, status of the tissue is evaluated from the monitored resonant frequency. Evaluating the status can include identifying changes in the monitored resonant frequency as a function of time and correlating the identified changes to a healing status of the tissue. Identifying the changes and correlating the identified changes can include identifying a shift of the monitored resonant frequency to lower frequencies with increase in time, from an initial interrogation of the resonant sensor with the patch attached to the tissue, as a healing of the tissue. In cases where a wound is in the form of an open cavity that fills in with fluid, going from a low air dielectric to a higher tissue dielectric, the resonant frequency can shift down. In wounds that begin with excess fluid, as this fluid is reduced, that is the tissue dries, which is accompanied by decreases in tissue dielectric, the resonant frequency can increase. The directional change in the resonant frequency can depend on the phase of the healing process and type of wound. The type of tissue being monitored can be used in evaluating the change in resonant frequency over time. A variety of tissue abnormalities (burns, open wounds, ulcers, inflammation, etc.) can change the tissue dielectric. If the tissue dielectric goes down, the resonant frequency can increase. If the tissue dielectric increases, the resonant frequency can decrease.

Variations of method 800 or methods similar to method 800 can include a number of different embodiments that can be combined depending on the application of such methods and/or the architecture of systems in which such methods are implemented. Such methods can include scanning the resonant sensor to measure changes in morphology of the tissue, using the set of antennas and network analyzer to detect a phase and a magnitude of each of a S11 scattering parameter and a S21 scattering parameter. Method 800 and methods similar to method 800 can include features associated with any of FIGS. 1-7. Method 800 and methods similar to the method 800 can also include features associated with a passive patch-resonant sensor structure as taught herein.

In various embodiments, a non-transitory machine-readable storage device, such as computer-readable non-transitory media, can comprise instructions stored thereon, which, when performed by a machine, cause the machine to perform operations, where the operations comprise one or more features similar to or identical to features of methods and techniques described with respect to method 800, variations thereof, and/or features of other methods taught herein such as associated with FIGS. 1-7. The physical structures of such instructions may be operated on by one or more processors. For example, executing these physical structures can cause the machine to perform operations comprising operations to: interrogate, at a number of different times by use of a set of antennas and a network analyzer, a resonant sensor attached to or integrated into a patch with the patch attached to tissue, the patch and resonant sensor structured such that, when contacting the tissue, dielectric of the contacted tissue contributes to capacitance of the resonant sensor to affect a resonant frequency of the resonant sensor; monitor the resonant frequency of the resonant sensor from the interrogation at the number of different times; and evaluate status of the tissue from the monitored resonant frequency.

Operations to evaluate the status of the tissue can include operations to identify changes in the monitored resonant frequency as a function of time and to correlate the identified changes to a healing status of the tissue. Operations to identify changes and to correlate the identified changes can include identification of a shift of the monitored resonant frequency to lower frequencies with increase in time, from an initial interrogation of the resonant sensor with the patch attached to the tissue, as a healing of the tissue. In cases where a wound is in the form of an open cavity that fills in with fluid, going from a low air dielectric to a higher tissue dielectric, the resonant frequency can shift down. In wounds that begin with excess fluid, as this fluid is reduced, that is the tissue dries, which is accompanied by decreases in tissue dielectric, the resonant frequency can increase. The directional change in the resonant frequency can depend on the phase of the healing process and type of wound. The type of tissue being monitored can be used in evaluating the change in resonant frequency over time. A variety of tissue abnormalities (burns, open wounds, ulcers, inflammation, etc.) can change the tissue dielectric. If the tissue dielectric goes down, the resonant frequency can increase. If the tissue dielectric increases, the resonant frequency can decrease. Operations can include operations to scan the resonant sensor to measure changes in morphology of the tissue, using the set of antennas and network analyzer to detect a phase and a magnitude of each of a S11 scattering parameter and a S21 scattering parameter.

Further, machine-readable storage devices, such as computer-readable non-transitory media, herein, are physical devices that stores data represented by physical structure within the respective device. Such a physical device is a non-transitory device. Examples of machine-readable storage devices can include, but are not limited to, read only memory (ROM), random access memory (RAM), a magnetic disk storage device, an optical storage device, a flash memory, and other electronic, magnetic, and/or optical memory devices. The machine-readable device may be a machine-readable medium such as storage device 235 of FIG. 2. While storage device 235 is shown as a single component unit, terms such as “memory,” “machine-readable medium,” “machine-readable device,” and similar terms should be taken to include all forms of storage media, either in the form of a single medium (or device) or multiple media (or devices), in all forms. For example, such structures can be realized as centralized database(s), distributed database(s), associated caches, and servers; one or more storage devices, such as storage drives (including but not limited to electronic, magnetic, and optical drives and storage mechanisms), and one or more instances of memory devices or modules (whether main memory; cache storage, either internal or external to a processor; or buffers). Terms such as “memory,” “storage device,” “machine-readable medium,” and “machine-readable device.” shall be taken to include any tangible non-transitory medium which is capable of storing or encoding a sequence of instructions for execution by the machine and that cause the machine to perform any one of the methodologies taught herein. The term “non-transitory” used in reference to a “machine-readable device,” “medium,” “storage medium,” “device,” or “storage device” expressly includes all forms of storage drives (optical, magnetic, electrical, etc.) and all forms of memory devices (e.g., DRAM, Flash (of all storage designs), SRAM, MRAM, phase change, etc., as well as all other structures designed to store data of any type for later retrieval.

In various embodiments, an apparatus can comprise a resonant sensor having an inductive element and a capacitive element, and a patch to which the resonant sensor is attached or in which the resonant sensor is integrated, with the patch being attachable to tissue. The patch and resonant sensor can be structured such that, when contacting the tissue, dielectric of the contacted tissue contributes to capacitance of the resonant sensor to affect a resonant frequency of the resonant sensor.

Variations of the apparatus or similar apparatus can include a number of different embodiments that can be combined depending on the application of such apparatus and/or the architecture in which such apparatus are implemented. In such apparatus, the resonant sensor can be flexible with the inductive element structured as an electrically conductive coil on a polymer film. The capacitive element can include dielectric material between conductive lines of the electrically conductive coil. The electrically conductive coil can include copper.

Variations of the apparatus can include the inductive element being a conductive trace material with the conductive trace material encapsulated. The apparatus can be a bandage to protect the tissue to which the bandage is attached during a healing process of the tissue.

In various embodiments, a system can comprise: a resonant sensor having an inductive element and a capacitive element; a patch to which the resonant sensor is attached or in which the resonant sensor is integrated, with the patch being attachable to tissue, the patch and resonant sensor structured such that, when contacting the tissue, dielectric of the contacted tissue contributes to capacitance of the resonant sensor to affect a resonant frequency of the resonant sensor; a set of antennas: and a network analyzer to couple to the set of antennas to interrogate the resonant sensor.

Variations of the system or similar systems can include a number of different embodiments that can be combined depending on the application of such systems and/or the architecture in which such systems are implemented. In such a system, the resonant sensor can be flexible with the inductive element structured as an electrically conductive coil on a polymer film. Such variations can include the inductive element being a conductive trace material with the conductive trace material encapsulated. The resonant sensor and the patch can be structured together as a bandage to protect the tissue to which the bandage is attached during a healing process of the tissue.

Variations of the system or similar systems can include one or more processors; and a storage device comprising instructions, which when executed by the one or more processors, cause the system to perform operations to: interrogate the resonant sensor, with the patch attached to tissue, at a number of different times using the set of antennas and the network analyzer; monitor the resonant frequency of the resonant sensor from the interrogation at each time of the number of different times; and evaluate status of the tissue from the monitored resonant frequencies. The operations to evaluate the status of the tissue can include operations to identify changes in the monitored resonant frequency as a function of time and to correlate the identified changes to a healing status of the tissue. The operations to identify changes and to correlate the identified changes can include identification of a shift of the monitored resonant frequency to lower frequencies with increase in time, from an initial interrogation of the resonant sensor with the patch attached to the tissue, as a healing of the tissue. In cases where a wound is in the form of an open cavity that fills in with fluid, going from a low air dielectric to a higher tissue dielectric, the resonant frequency can shift down. In wounds that begin with excess fluid, as this fluid is reduced, that is the tissue dries, which is accompanied by decreases in tissue dielectric, the resonant frequency can increase. The directional change in the resonant frequency can depend on the phase of the healing process and type of wound. The type of tissue being monitored can be used in evaluating the change in resonant frequency over time. A variety of tissue abnormalities (burns, open wounds, ulcers, inflammation, etc.) can change the tissue dielectric. If the tissue dielectric goes down, the resonant frequency can increase. If the tissue dielectric increases, the resonant frequency can decrease. Operation of such systems can include operations to scan the resonant sensor to measure changes in morphology of the tissue, using the set of antennas and network analyzer to detect a phase and a magnitude of each of a S11 scattering parameter and a S21 scattering parameter.

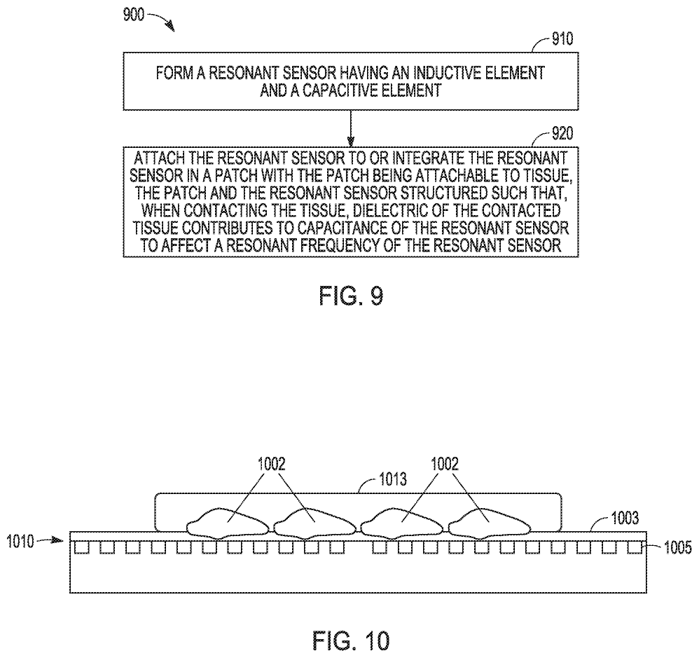

FIG. 9 is a flow diagram of features of an embodiment of an example method 900 of forming a passive patch-resonant sensor structure. At 910, a resonant sensor is formed having an inductive element and a capacitive element. Forming the resonant sensor can include: forming, with a masking material, a resonator design on a polymer coated with a conductive material; etching the polymer coated with the conductive material having the formed resonator design; and after etching, washing off the masking material to form a conductive coil. The conductive material can be copper. The coil can be an Archimedean coil.

At 920, the resonant sensor is attached to or integrated into a patch with the patch being attachable to tissue. The patch and the resonant sensor can be structured such that, when contacting the tissue, dielectric of the contacted tissue contributes to capacitance of the resonant sensor to affect a resonant frequency of the resonant sensor.

Variations of the method 900 or methods similar to method 900 can include a number of different embodiments that can be combined depending on the application of such methods and/or the architecture of systems in which such methods are implemented. Such methods can include encapsulating the coil. Such methods can include forming the patch and the resonant sensor as a bandage to protect the tissue to which the bandage is attached during a healing process of the tissue.

FIG. 10 illustrates an example resonant sensor 1010 with coil elements 1005 implemented in experiments in order to investigate the capability of the resonant sensor to detect bacteria 1002 on a surface. The experiment arrangement includes bacteria 1002 on a polyimide (insulated) side 1003 of the resonant sensor 1010 with an agar plate 1013 on the bacteria 1002.

Using the resonant sensor 1010 of FIG. 10, a preliminary study was conducted in which the sensor response to varying concentrations of Bacillus subtilis (B. subtilis) was monitored for 12 hours. First, lysogeny broth (LB) medium was prepared by dissolving 10 g tryptone. 5 g yeast extract, and 10 g sodium chloride (NaCl) in 1000 ml deionized water. To sterilize, the solution was autoclaved on liquid cycle for 60 minutes and then stored in the fridge (4° C.). For preparing the bacteria solution. 3 ml LB. 3 μl IPTG, and 3 μl CHM were added to a sterilized culture tube. A small amount of B. subtilis was added to the solution using a 1000 μl pipette tip. The sample was briefly vortexed, and the culture tube was placed at an angle in a shaker incubator for overnight growth at 37° C. for 12 hours. Afterward, the optical density (OD) was measured and the cells were diluted with fresh LB to OD=1.

Next. 25 μl of bacteria solution was placed on the polyimide (insulated) side 1003 of the resonant sensor 1010, which was secured on the reader antenna, and placed at a 37C incubator. The agar plate 1013 was placed on top of the B. subtilis suspension; therefore, the cells were sandwiched between the agar plate 1013 and insulated side 1003 of the sensor 1010, eliminating the evaporation. The evaporation was further eliminated by creating a small humidity chamber surrounding the resonator system. The resonant sensor used for this experiment had an inner diameter of 1.5 mm, an outer diameter of 40 mm, and a pitch size of 1.2 mm. An increase in signal is observed.

The agar plate 1013 was prepared by adding 2 g agar to 100 ml of LB solution. The mixture was autoclaved again on liquid cycle for 60 minutes and then kept at 50° C. on a hot plate for sample preparation. A silicone gasket flat washer was vacuum greased on a glass microscope slide to act as a sealing ring. Afterward, 300 μl of agar media was placed within the washer and gelled after 2 minutes. Next, the washer was carefully removed, and the agar was carefully placed on the cell culture, on the resonant sensor.

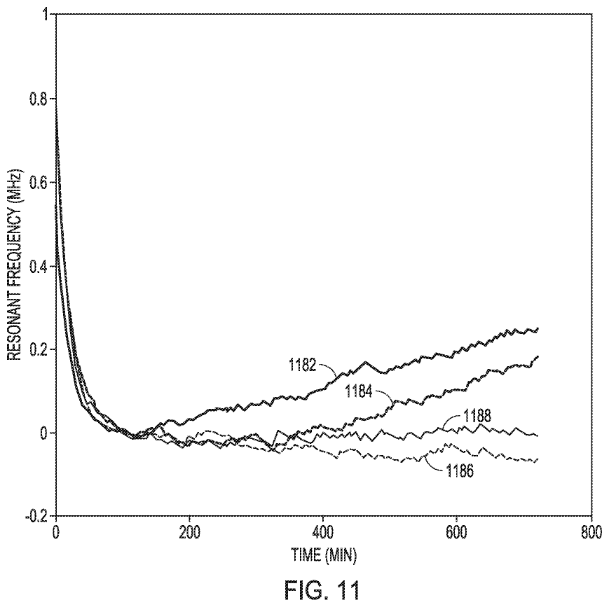

The resonator sensor 1010 was used in an arrangement similar to the reading and evaluating arrangement of system 200 of FIG. 2. FIG. 11 shows experimental results in a plot of resonant frequency versus time for several OD dilutions of B. subtilis cell culture. Curve 1182 is a result for an OD=1 dilution of B. subtilis cell culture. Curve 1184 is a result for an OD ten times (10×) dilution of B. subtilis cell culture. Curve 1186 is a result for an OD hundred times (100×) dilution of B. subtilis cell culture. Curve 1188 is a result for an OD thousand times (1000×) dilution of B. subtilis cell culture. In order to make the dilution solutions, the B. subtilis cell culture with OD=1 was further diluted with fresh LB media. For each, an increase in signal is observed. By monitoring the resonant frequency of the sensor for 12 hours, it was observed that the sensor is able to respond to different levels of bacteria growth on a surface, which can be used for detecting wound infection when embedded in a dressing patch.

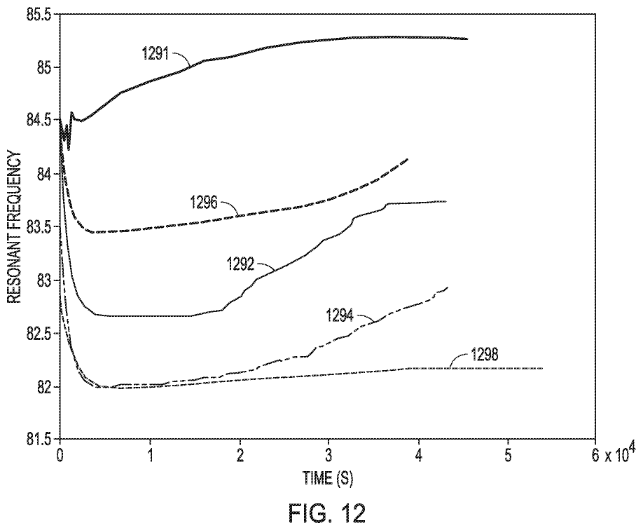

FIG. 12 shows additional experimental results in a plot of resonant frequency versus time for several OD dilutions of B. subtilis cell culture as well as LB control. Curve 1291 is a result for an OD=0.56 dilution of B. subtilis cell culture. Curve 1292 is a result for an OD ten times (10×) dilution of B. subtilis cell culture. Curve 1294 is a result for an OD hundred times (100×) dilution of B. subtilis cell culture. Curve 1296 is a result for an OD thousand times (1000×) dilution of B. subtilis cell culture. Curve 1298 is a result for a control media only.

The following are example embodiments of methods, apparatus, and systems, in accordance with the teachings herein.

An example apparatus 1 can comprise: a resonant sensor having an inductive element and a capacitive element; and a patch to which the resonant sensor is attached or in which the resonant sensor is integrated, with the patch being attachable to tissue, the patch and resonant sensor structured such that, when contacting the tissue, dielectric of the contacted tissue contributes to capacitance of the resonant sensor to affect a resonant frequency of the resonant sensor.

An example apparatus 2 can include elements of example apparatus 1, wherein the resonant sensor is flexible with the inductive element structured as an electrically conductive coil on a polymer film.

An example apparatus 3 can include elements of any preceding example apparatus, wherein the capacitive element includes dielectric material between conductive lines of the electrically conductive coil.

An example apparatus 4 can include elements of any preceding example apparatus, wherein the electrically conductive coil includes copper.

An example apparatus 5 can include elements of any preceding example apparatus, wherein the inductive element is conductive trace material with the conductive trace material encapsulated.

An example apparatus 6 can include elements of any preceding example apparatus, wherein the apparatus is a bandage to protect the tissue to which the bandage is attached during a healing process of the tissue.

An example system 1 can comprise an apparatus of any of the preceding example apparatus.

An example system 2 can comprise: a resonant sensor having an inductive element and a capacitive element; a patch to which the resonant sensor is attached or in which the resonant sensor is integrated, with the patch being attachable to tissue, the patch and resonant sensor structured such that, when contacting the tissue, dielectric of the contacted tissue contributes to capacitance of the resonant sensor to affect a resonant frequency of the resonant sensor; a set of antennas; and a network analyzer to couple to the set of antennas to interrogate the resonant sensor.

An example system 3 can include elements of example system 2, wherein the resonant sensor is flexible with the inductive element structured as an electrically conductive coil on a polymer film.

An example system 4 can include elements of any preceding example systems, wherein the inductive element is conductive trace material with the conductive trace material encapsulated.

An example system 5 can include elements of any preceding example systems, wherein the resonant sensor and the patch are structured together as a bandage to protect the tissue to which the bandage is attached during a healing process of the tissue.

An example system 6 can include elements of any preceding example systems, wherein the system includes: one or more processors; and a storage device comprising instructions, which when executed by the one or more processors, cause the system to perform operations to: interrogate the resonant sensor, with the patch attached to tissue, at a number of different times using the set of antennas and the network analyzer; monitor the resonant frequency of the resonant sensor from the interrogation at each time of the number of different times; and evaluate status of the tissue from the monitored resonant frequencies.

An example system 7 can include elements of any preceding example systems, wherein the operations to evaluate the status of the tissue include operations to identify changes in the monitored resonant frequency as a function of time and to correlate the identified changes to a healing status of the tissue.

An example system 8 can include elements of any preceding example systems, wherein the operations to identify changes and to correlate the identified changes include identification of a shift of the monitored resonant frequency to lower frequencies with increase in time, from an initial interrogation of the resonant sensor with the patch attached to the tissue, as a healing of the tissue.

An example system 9 can include elements of any preceding example systems, wherein the operations include operations to scan the resonant sensor to measure changes in morphology of the tissue, using the set of antennas and network analyzer to detect a phase and a magnitude of each of a S11 scattering parameter and a S21 scattering parameter.

An example method 1 can comprise operating any example apparatus 1-6.

An example method 2 can comprise forming any example apparatus 1-6.

An example method 3 can comprise operating any example system 1-9.

An example method 4 can comprise forming any example system 1-9.

An example method 5 can comprise: interrogating, at a number of different times using a set of antennas and a network analyzer, a resonant sensor attached to or integrated into a patch with the patch attached to tissue, the patch and resonant sensor structured such that, when contacting the tissue, dielectric of the contacted tissue contributes to capacitance of the resonant sensor to affect a resonant frequency of the resonant sensor; monitoring the resonant frequency of the resonant sensor from the interrogation at the number of different times; and evaluating status of the tissue from the monitored resonant frequency.

An example method 6 can include elements of preceding example method 5, wherein evaluating the status includes identifying changes in the monitored resonant frequency as a function of time and correlating the identified changes to a healing status of the tissue.

An example method 7 can include elements of any preceding example methods 5 and 6, wherein identifying the changes and correlating the identified changes includes identifying a shift of the monitored resonant frequency to lower frequencies with increase in time, from an initial interrogation of the resonant sensor with the patch attached to the tissue, as a healing of the tissue.

An example method 8 can include elements of any preceding example methods 5-7, wherein the method includes scanning the resonant sensor to measure changes in morphology of the tissue, using the set of antennas and network analyzer to detect a phase and a magnitude of each of a S11 scattering parameter and a S21 scattering parameter.

An example machine-readable storage device comprising instructions, which, when executed by a set of processors, cause a system to perform operations, the operations comprising operations to perform elements of any of example methods 1-8.

An example method 9 can comprise: forming a resonant sensor having an inductive element and a capacitive element: and attaching the resonant sensor to or integrating the resonant sensor in a patch with the patch being attachable to tissue, the patch and the resonant sensor structured such that, when contacting the tissue, dielectric of the contacted tissue contributes to capacitance of the resonant sensor to affect a resonant frequency of the resonant sensor.

An example method 10 can include elements of example method 9, wherein forming the resonant sensor includes: forming, with a masking material, a resonator design on a polymer coated with a conductive material: etching the polymer coated with the conductive material having the formed resonator design; and after etching, washing off the masking material to form a conductive coil.

An example method 11 can include elements of any preceding example methods 9 and 10, wherein the conductive material is copper.

An example method 12 can include elements of any preceding example methods 9-11, wherein the coil is an Archimedean coil.

An example method 13 can include elements of any preceding example methods 9-12, wherein the method includes encapsulating the coil.

An example method 14 can include elements of any preceding example methods 9-13, wherein the method includes forming the patch and the resonant sensor as a bandage to protect the tissue to which the bandage is attached during a healing process of the tissue.

A robust system for wireless measurement of wound health, as taught herein, can find many impactful ready applications. Such a robust system may be used in at-home monitoring of wound health, which may reduce frequency of clinic visits. Such a robust system may be used in detecting onset and monitoring of lower extremity complications from diabetes. Such a robust system may be used as consumer, in-bandage sensors to monitor wound healing in general.

Although specific embodiments have been illustrated and described herein, it will be appreciated by those of ordinary skill in the art that any arrangement that is calculated to achieve the same purpose may be substituted for the specific embodiments shown. Various embodiments use permutations and/or combinations of embodiments described herein. It is to be understood that the above description is intended to be illustrative, and not restrictive, and that the phraseology or terminology employed herein is for the purpose of description.

What is claimed is:

1. An apparatus comprising:
a resonant sensor having an inductive element and a capacitive element; and
a patch to which the resonant sensor is attached or in which the resonant sensor is integrated, with the patch being attachable to tissue, the patch and resonant sensor structured such that, when contacting the tissue, dielectric of the contacted tissue contributes to capacitance of the resonant sensor to affect a resonant frequency of the resonant sensor.

2. The apparatus of claim 1, wherein the resonant sensor is flexible with the inductive element structured as an electrically conductive coil on a polymer film.

3. The apparatus of claim 2, wherein the capacitive element includes dielectric material between conductive lines of the electrically conductive coil.

4. The apparatus of claim 1, wherein the inductive element is conductive trace material with the conductive trace material encapsulated.

5. The apparatus of claim 1, wherein the apparatus is a bandage to protect the tissue to which the bandage is attached during a healing process of the tissue.

6. A system comprising:
a resonant sensor having an inductive element and a capacitive element;
a patch to which the resonant sensor is attached or in which the resonant sensor is integrated, with the patch being attachable to tissue, the patch and resonant sensor structured such that, when contacting the tissue, dielectric of the contacted tissue contributes to capacitance of the resonant sensor to affect a resonant frequency of the resonant sensor;
a set of antennas; and
a network analyzer to couple to the set of antennas to interrogate the resonant sensor.

7. The system of claim 6, wherein the resonant sensor is flexible with the inductive element structured as an electrically conductive coil on a polymer film.

8. The system of claim 6, wherein the resonant sensor and the patch are structured together as a bandage to protect the tissue to which the bandage is attached during a healing process of the tissue.

9. The system of claim 6, wherein the system includes:
one or more processors; and
a storage device comprising instructions, which when executed by the one or more processors, cause the system to perform operations to:
interrogate the resonant sensor, with the patch attached to tissue, at a number of different times using the set of antennas and the network analyzer;
monitor the resonant frequency of the resonant sensor from the interrogation at each time of the number of different times; and
evaluate status of the tissue from the monitored resonant frequencies.

10. The system of claim 9, wherein the operations to evaluate the status of the tissue include operations to identify changes in the monitored resonant frequency as a function of time and to correlate the identified changes to a healing status of the tissue.

11. The system of claim 10, wherein the operations to identify changes and to correlate the identified changes include identification of a shift of the monitored resonant frequency to lower frequencies with increase in time, from an initial interrogation of the resonant sensor with the patch attached to the tissue, as a healing of the tissue.

12. The system of claim 9, wherein the operations include operations to scan the resonant sensor to measure changes in morphology of the tissue, using the set of antennas and network analyzer to detect a phase and a magnitude of each of a S11 scattering parameter and a S21 scattering parameter.

13. A method comprising:
interrogating, at a number of different times using a set of antennas and a network analyzer, a resonant sensor attached to or integrated into a patch with the patch attached to tissue, the patch and resonant sensor structured such that, when contacting the tissue, dielectric of the contacted tissue contributes to capacitance of the resonant sensor to affect a resonant frequency of the resonant sensor;
monitoring the resonant frequency of the resonant sensor from the interrogation at the number of different times; and
evaluating status of the tissue from the monitored resonant frequency.

14. The method of claim 13, wherein evaluating the status includes identifying changes in the monitored resonant frequency as a function of time and correlating the identified changes to a healing status of the tissue.

15. The method of claim 14, wherein identifying the changes and correlating the identified changes includes identifying a shift of the monitored resonant frequency to lower frequencies with increase in time, from an initial interrogation of the resonant sensor with the patch attached to the tissue, as a healing of the tissue.

16. A machine-readable storage device comprising instructions, which, when executed by a set of processors, cause a system to perform operations, the operations comprising operations to:
interrogate, at a number of different times by use of a set of antennas and a network analyzer, a resonant sensor attached to or integrated into a patch with the patch attached to tissue, the patch and resonant sensor structured such that, when contacting the tissue, dielectric of the contacted tissue contributes to capacitance of the resonant sensor to affect a resonant frequency of the resonant sensor;
monitor the resonant frequency of the resonant sensor from the interrogation at the number of different times; and
evaluate status of the tissue from the monitored resonant frequency.

17. The machine-readable storage device of claim 16, wherein operations to evaluate the status of the tissue include operations to identify changes in the monitored resonant frequency as a function of time and to correlate the identified changes to a healing status of the tissue.

18. The machine-readable storage device of claim 16, wherein the operations include operations to scan the resonant sensor to measure changes in morphology of the tissue, using the set of antennas and network analyzer to detect a phase and a magnitude of each of a S11 scattering parameter and a S21 scattering parameter.

19. A method comprising:
forming a resonant sensor having an inductive element and a capacitive element; and
attaching the resonant sensor to or integrating the resonant sensor in a patch with the patch being attachable to tissue, the patch and the resonant sensor structured such that, when contacting the tissue, dielectric of the contacted tissue contributes to capacitance of the resonant sensor to affect a resonant frequency of the resonant sensor.

20. The method of claim 19, wherein forming the resonant sensor includes:
forming, with a masking material, a resonator design on a polymer coated with a conductive material;
etching the polymer coated with the conductive material having the formed resonator design; and
after etching, washing off the masking material to form a conductive coil.
